(19) United States
(12) Patent Application Publication
(10) Pub. No.: US 2011/0008901 A1
(43) Pub. Date: Jan. 13, 2011

(54) APOLIPOPROTEIN CIII IN PRE- AND TYPE 2 DIABETES

(76) Inventors: Urban A. Kiernan, Chandler, AZ (US); Dobrin Nedelkov, Tempe, AZ (US)

Correspondence Address: Zeman-Mullen Law Firm PLLC, 233 White Spar Road, Prescott, AZ, 86303, US

(21) Appl. No.: 12/829,891

(22) Filed: Jul. 2, 2010

Related U.S. Application Data
(60) Provisional application No. 61/223,502, filed on Jul. 7, 2009.

(51) Int. Cl. G01N 33/48; G01N 27/447; G01N 33/483; G01N 1/30; G01N 30/72; G01N 21/75

(52) U.S. Cl. 436/86

(57) ABSTRACT

The present invention is directed to diagnosing, determining, and/or monitoring type 2 diabetes, pre-diabetes, insulin resistance, and their related conditions by detecting levels and modulations of ApoCIII and its variants. The present invention is also directed to methods for identifying and evaluating therapeutic treatments for type 2 diabetes, pre-diabetes, insulin resistance, and their related conditions by monitoring ApoCIII and its variants.

### CROSS-REFERENCE TO RELATED APPLICATIONS

This application claims priority to provisional patent application having Ser. No. 61/223,502 filed Jul. 7, 2009 which is herein incorporated by reference in its entirety.

### FIELD OF THE INVENTION

This invention relates to methods for determining, diagnosing, and/or assessing pre-diabetes, type 2 diabetes, insulin resistance and their related conditions by detecting levels and modulations of Apolipoprotein CIII (ApoCIII) and its associated variants. This invention also relates to methods for screening molecules that modulate ApoCIII and its associated variants and their use in the treatment and/or amelioration of symptoms that are related to pre-diabetes, type 2 diabetes, insulin resistance and their related conditions.

### BACKGROUND OF THE INVENTION

Type 2 diabetes (also known as diabetes mellitus type 2, non-insulin-dependent diabetes mellitus or adult on-set diabetes) is a metabolic disorder that is characterized by high blood glucose. This condition is a result of either a lack of insulin produced by the body or the developed tolerance to insulin by the cells of the body (Insulin Resistance). Since insulin is essential for the absorption of glucose from the blood, perturbations in insulin homeostasis eventually result in loss of glycemic control and the development of the diabetic condition. If left uncontrolled, excessive blood glucose will eventually lead to a multitude of complications, including but not limited to: blindness, skin ulcerations, amputations, heart disease and kidney disease. Many proposed theories exist regarding the association of multiple complications that are linked to the loss of glycemic control, however, the mechanisms in which these multiple pathways interact is still unknown.

As of 2007, the Centers of Disease Control determined that 7.8% of the US population (23.6 million people) have type 2 diabetes and it is estimated that >20% of Americans are pre-diabetic. Due to the debilitating nature of these conditions and their associated cost of treatment, more effective and efficient methods for earlier detection are paramount to the health of the US populace and western civilization. The “gold standard” for evaluating the level of glycemic control is the Oral Glucose Tolerance Test (OGTT). This test involves measuring the fasting blood glucose of an individual or patient and then obtaining a blood glucose measurement 2 hours post administration of an oral glucose solution. Current guidelines define normal glucose tolerance (NGT) as a 2 hour glucose level≦140 mg/dl. The criteria for type 2 diabetes (DM) is a 2 hour glucose of ≧200 mg/dl and the definition of impaired glucose tolerance (IGT or pre-diabetes) is between 140 and 200 mg/dl. Even though these criteria are sanctioned by both the American Diabetes Association and the World Health Organization, these values are not without clinical error or subject to interpretation.

A recent development in diabetes care included the adoption of guidelines for the use of the protein biomarker hemoglobin A1C (HbA1C) in the diagnosis of diabetes and pre-diabetes. Due to the longevity of hemoglobin in the blood, it serves as a long term measure of glycemic control. Although long used as a biomarker of whether or not a patient's diabetes treatment is sufficient, conflicting data previously prevented the use of HbA1C in diabetes determination. Even with the acceptance of these new guidelines, and their use in conjunction with the standard OGTT criteria and fasting blood glucose levels, the diagnosis of pre-diabetes or type 2 diabetes still ultimately relies on the interpretation and the judgment of the diagnosing physician.

Due to the pandemic nature of type 2 diabetes, pre-diabetes, insulin resistance and the development of their associated complications, there is a world wide need for additional detection/diagnostic/assessment methods to allow for the earliest possible intervention and to provide a means to evaluate the effectiveness of treatment through life-style changes and/or medication. A need also exists for additional metabolic or endocrine targets for the development of treatments that alleviate or ameliorate the problems and symptoms associated with these related conditions.

### SUMMARY OF THE INVENTION

As shown here for the first time, modulations in the concentration of ApoCIII and its associated variants correlate with the 2 hour glucose values obtained after the application of an OGTT and the clinical diagnosis of certain metabolic disease states. These findings identify ApoCIII and its variants as a biomarker for the clinical assessment/diagnosis of type 2 diabetes, pre-diabetes, insulin resistance and their related conditions. It also stands to reason that ApoCIII and its variants may also mechanistically participate in the initiation/development and pathology of these disease states and therefore may also have utility as a therapeutic target for the amelioration and/or treatment of the disease symptoms or condition.

One objective of the present invention is to provide a new target and screening method for the analysis of ApoCIII in the in-vitro and in-vivo detection and/or diagnosis of type 2 diabetes, pre-diabetes, insulin resistance and their related conditions.

Another objective of the present invention is to use ApoCIII variants as new targets and screening methods for the in-vitro and in-vivo detection and/or diagnosis of type 2 diabetes, pre-diabetes, insulin resistance and their related conditions.

A further objective of the present invention is to use ApoCIII as a new target for molecules that modulate ApoCIII and/or in the screening of molecules aimed at the in-vitro and/or in-vivo modulation of its expression, activity and/or clearance in the treatment of type 2 diabetes, pre-diabetes, insulin resistance and their related conditions.

Yet a further objective of the present invention is to use ApoCIII variants as a new target for molecules that modulate ApoCIII variants and/or in the screening of molecules aimed at the in-vitro and/or in-vivo modulation of their expression, activity and/or clearance in the treatment of type 2 diabetes, pre-diabetes, insulin resistance and their related conditions.

Some novel features that are considered characteristic of the invention are set forth with particularity in the claims. The invention itself, however, both as to its structure and its operation together with the additional objectives and advantages thereof will best be understood from the following description of the exemplary embodiment of the present invention when read in conjunction with the accompanying drawings. Unless specifically noted, it is intended that the words and phrases in the specification and claims be given the ordinary and accustomed meaning for those of ordinary skill in the application of the art or arts. If any other meaning is intended, the specification will specifically state that special meaning is being applied to the word or phrase. Likewise, the use of the words “function” or “means” in the Description of Preferred Embodiments is not intended to indicate a desire to invoke the special provisions of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means or step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include all structures, materials or acts that perform the claimed function, along with any known or later-developed equivalent structures, materials or acts for performing the claimed function.

### BRIEF DESCRIPTION OF THE DRAWINGS

The object and features of the present invention will be better understood with reference to the following detailed description and drawings.

FIG. 1 illustrates representative mass spectral results of the analysis of ApoCIII and its isoforms and variants from human plasma samples using mass spectrometric immunoassay.

FIG. 2 is a dot histogram of the observed total ApoCIII abundance from the human plasma samples from patients with normal glucose tolerance (NGT) and patients with impaired glucose tolerance (IGT, pre-diabetes) that were analyzed and shown in FIG. 1.

FIG. 3 is a dot histogram of the observed total ApoCIII abundance from the human plasma samples from patients with normal glucose tolerance (NGT) and patients with type 2 diabetes (DM) that were analyzed and shown in FIG. 1.

FIG. 4 shows the resultant ROC curves established from the mass spectral total ApoCIII abundance data obtained from each sample population, namely the human plasma samples from patients with normal glucose tolerance (NGT), the human plasma samples from patients with impaired glucose tolerance (IGT, pre-diabetes), and the human plasma samples from patients with type 2 diabetes (DM), that were analyzed and shown in FIG. 1.

FIG. 5 is a dot histogram of the abundance of an ApoCIII variant, namely the observed total ApoCIII(1) abundance, from the human plasma samples from patients with normal glucose tolerance (NGT) and patients with impaired glucose tolerance (IGT, pre-diabetes) that were analyzed and shown in FIG. 1.

FIG. 6 shows a dot histogram of the observed total ApoCIII(1) abundance from the human plasma samples from patients with normal glucose tolerance (NGT) and patients with type 2 diabetes (DM) that were analyzed and shown in FIG. 1.

FIG. 7 shows the resultant ROC curves established from the mass spectral total ApoCIII(1) abundance data obtained from each sample population, namely the human plasma samples from patients with normal glucose tolerance (NGT), the human plasma samples from patients with impaired glucose tolerance (IGT, pre-diabetes), and the human plasma samples from patients with type 2 diabetes (DM), that were analyzed and shown in FIG. 1.

### DETAILED DESCRIPTION

It is demonstrated for the first time in the present invention that ApoCIII and its variants correlate with the clinical diagnosis of both the pre-diabetes and type 2 diabetes disease states. It is also demonstrated for the first time that the modulations in the concentration of ApoCIII and its variants compare to the results of the OGTT. This observed phenomenon directly displays the utility of this novel marker as it relates to type 2 diabetes, pre-diabetes, insulin resistance and/or their related conditions or complications.

The present invention encompasses the use of total ApoCIII as a biomarker for the in-vitro and/or in-vivo determination/diagnosis/assessment of type 2 diabetes, pre-diabetes, insulin resistance and/or their related conditions or complications. The present invention also includes the use of specific ApoCIII variant(s) for the same purpose.

In addition, the present invention includes the use of ApoCIII as a marker for the in-vitro and/or in-vivo screening of compounds, aimed at modulating the expression, activity and/or clearance of ApoCIII for the purpose of treating, or reducing symptoms that are associated with, pre-diabetes, type 2 diabetes, insulin resistance and/or their related conditions or complications. The present invention also includes the use of specific ApoCIII variant(s) for this same purpose.

The present invention also includes methodologies for the measurement of ApoCIII and its variants from biological samples for the determination/diagnosis/assessment of type 2 diabetes, pre-diabetes, insulin resistance and/or their related conditions or complications.

The present invention also extends these methodologies for the measurement of ApoCIII and its variants in order to screen compounds, aimed at modulating the expression, activity and/or clearance of ApoCIII and its variants for the purpose of treating, or reducing symptoms that are associated with, pre-diabetes, type 2 diabetes, insulin resistance and/their related conditions or complications.

### DEFINITIONS

All the technical and scientific terms used herein, unless defined otherwise, have the meaning commonly understood by a person skilled in art to which this invention pertains. As used herein, the following terms have the meaning ascribed to them unless specified otherwise.

As used herein, “ApoCIII” is a general term describing an apolipoprotein expressed by the gene APOC3. It constitutes 50% of the protein fraction of VLDL and 2% of that of HDL. Its known function is to inhibit lipoprotein lipase and hepatic lipase. It has previously been shown that elevations in ApoCIII levels induce the development of hypertriglyceridemia.

As used herein, “variant” refers to different iso-forms or sub-forms of ApoCIII found in biological samples. Such sub-forms may be the result of truncation of amino acids from the primary structure, alterations or absence of sugars in the associated glycan, oxidation adducts or expressed genetic mutations.

As used herein, “ApoCIII(0)” refers to the specific ApoCIII variant in which the terminal sialic acid residues on the glycan structure are absent.

As used herein, “ApoCIII(1)” refers to the specific ApoCIII variant in which there is only a single terminal sialic acid residue on the glycan structure.

As used herein, “ApoCIII(2)” refers to the specific ApoCIII variant in which both the terminal sialic acid residues are present on the glycan structure.

As used herein, “analysis” refers to the measurement of the activity or concentration of ApoCIII and/or its variants from a biological sample.

As used herein, “biological sample” refers to a fluid or extract having a plurality of components. Complex media may include, but is not limited to, tissue, cell extracts, nuclear extracts, cell lysates and excretions, blood, sera, plasma, saliva, urine, sputum, synovial fluid, cerebral-spinal fluid, tears, feces, saliva, membrane extracts, industrial fluids and the like.

As used herein, “type 2 diabetes (DM)” is a clinical condition defined by excess glucose in the blood. Reference limits that aid in the clinical determination are a Fasting glucose value≧126 mg/dl and/or a 2 hour oral glucose tolerance test value≧200 mg/dl.

As used herein, “pre-diabetes, impaired glucose tolerance (IGT)” is a clinical condition defined by above normal amounts of glucose present in the blood. Reference limits that aid in the clinical determination are Fasting glucose values that are between 110 and 125 mg/dl and/or 2 hour oral glucose tolerance test values that are between 140 and 199 mg/dl.

As used herein, “normal glucose tolerance (NGT)” is a clinical determination when blood glucose levels are within normal concentration ranges. Reference limits that aid in the clinical determination are a Fasting glucose value<110 mg/dl and/or a 2 hour oral glucose tolerance test value<140 mg/dl.

As used herein, “insulin resistance” is a physical condition in which the hormone insulin becomes less effective in reducing blood glucose levels. The gold standard for evaluating insulin resistance is the administration of a hyperinsulinemic euglycemic clamp.

As used herein, “related conditions or complications” is defined as a variety of ailments that are commonly associated with the development and progression of type 2 diabetes. Some of these ailments include but are not limited to: heart attacks, skin ulcerations, blindness, amputations and kidney failure.

As used herein, “mass spectrometry” refers to the ability to volatilize/ionize analytes to form vapor-phase ions and determine their absolute or relative molecular masses. Suitable forms of volatilization/ionization are laser/light, thermal, electrical, atomized/sprayed and the like or combinations thereof. Suitable forms of mass spectrometry include, but are not limited to, Matrix Assisted Laser Desorption/Time of Flight Mass Spectrometry (MALDI-TOF MS), electrospray (or nanospray) ionization (ESI) mass spectrometry, or the like or combinations thereof.

As used herein, “subject” refers to any human, animal, or other living organism which possesses measurable levels of ApoCIII and/or ApoCIII variants in a biological sample taken there from.

In the present invention, the method for the measurement of activity or concentration of ApoCIII and/or its variants is performed using a biological sample (biological matrix). Possible biological matrices include, but are not limited to, tissue, whole blood, serum, plasma, saliva, cerebral spinal fluid or urine. The sample may also be free or immobilized onto a solid support. Examples of solid supports include, but are not limited to, filter paper, beads, arrays, etc. Any solid support material known in the art for immobilizing samples may be used. The sample may also be native in form or have undergone processing including, but not limited to, denaturation, reduction, proteolytic digestion and the like.

In order to measure ApoCIII and/or its variants, the present invention may utilize a separation or purification step to isolate or purify the biomarker from the biological sample. This separation can be performed by several means including, but not limited to, chromatography, centrifugation, affinity interactions, chemical methodologies, staining methodologies, and gel electrophoresis. Chromatography techniques may include but are not limited to: high performance liquid chromatography (HPLC), thin layer chromatography (TLC), paper chromatography (PC), affinity chromatography, size exclusion, ion-exchange, reverse phase, etc. Affinity interactions employ the ability of a molecule to bind with another molecule having proper fitting conformation. Affinity methods employed may include, but are not limited to, affinity interactions utilizing antibodies (Ab), antibody fragments (FAb), aptamers, proteins, peptides, lectins, etc. For sandwich assays, combinations of primary and secondary affinity ligands may be used. Gel electrophoresis methods may include, but are not limited to, acrylamide, agarose, etc. Chemical methodologies may include, but are not limited to, amino acid analysis, sequencing, etc. Staining methods may include, but are not limited to, the use of any dye, and the like, that directly binds to the ApoCIII protein and its variants, or to molecules that bind to ApoCIII and its variants.

The method for ApoCIII and/or ApoCIII variant measurement will utilize a detection technology for the isolated biomarker (i.e. the isolated ApoCIII and/or ApoCIII variant(s)). Detection may be direct or indirect in nature. Detection technologies include but are not limited to: staining, spectrometroscopy, spectrophotometry, colorimetry, fluorescence, luminescence, magnetism, radioisotopes, nuclear magnetic resonance spectroscopy, x-ray crystallography, surface plasmon resonance, mass spectrometry, etc. Depending on the state of the biological sample, the ApoCIII or ApoCIII variant form detected may be intact or a fragment thereof.

### Analyses Demonstrating Biomarker Utility

### Example

### Analysis of Apolipoprotein CIII and its Variants from Human Plasma

One exemplary embodiment of the invention is the concentration measurement of ApoCIII and its variants for use as a biomarker(s) to differentiate between clinically defined populations of samples. The analysis of this exemplary embodiment was performed using mass spectrometric immunoassay (MSIA) technology.

In this example, the analyses were performed on human citrate plasma samples obtained from patients that were newly diagnosed and clinically defined as having NGT, IGT or DM. There were 360 plasma samples obtained from NGT individuals, 98 plasma samples from patients with IGT, and 25 plasma samples from patients with DM which were all analyzed in the same manner.

The MSIA was performed with the aid of affinity pipette tips. The affinity pipette tips are made of small, porous microcolumns that are fitted at the entrance of a pipettor tip. The microcolumns are derivatized with an affinity ligand. The affinity ligand used to derivatize the affinity pipettes was anti-ApoCIII antibody. The sample preparation required the addition of an internal reference standard (IRS) for the semi-quantitative measurement of the ApoCIII. Each sample mixture consisted of 25 μL of ten-fold diluted (with buffer) human plasma, 30 μL of cynomologus monkey plasma (the cynomologus monkey ApoCIII is the IRS) and 145 μL of HEPES buffered saline with 1% tween 20. The prepared samples were interrogated with the anti-ApoCIII affinity pipette tips by repetitive drawing of the samples through the affinity pipettes. The purification process used HEPES buffered saline and water rinses following the ApoCIII affinity retrieval. Enriched and purified proteins were then eluted from the affinity pipettes with sinapic acid MALDI matrix and were deposited directly on a MALDI mass spectrometer target for subsequent mass spectral detection.

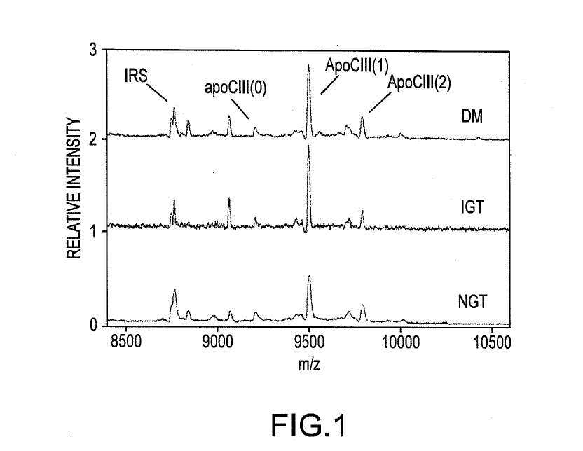

Examination of the resultant mass spectra showed signals from multiple forms of ApoCIII that are present in human plasma, as well as the IRS. Each spectrum was normalized to the integral of the ApoCIII signal obtained from the IRS. Within each mass spectrum, the mass shifted variants of human ApoCIII are clearly separated, with the ApoCIII(1) isoform being the most abundant in the majority of samples. Multiple ApoCIII variants were detected from each sample, but this example focuses on the three most abundant forms; namely ApoCIII(0), ApoCIII(1) and ApoCIII(2). The normalized spectra clearly show that ApoCIII(0), ApoCIII(1), and ApoCIII(2) modulate between the different samples. FIG. 1 illustrates that the abundance of ApoCIII and its variants is different in human plasma samples from patients with normal glucose tolerance (NGT), impaired glucose tolerance (IGT, pre-diabetes), and type 2 diabetes (DM). FIG. 1 shows that there is an increase in all ApoCIII variants in samples from patients diagnosed with IGT and DM.

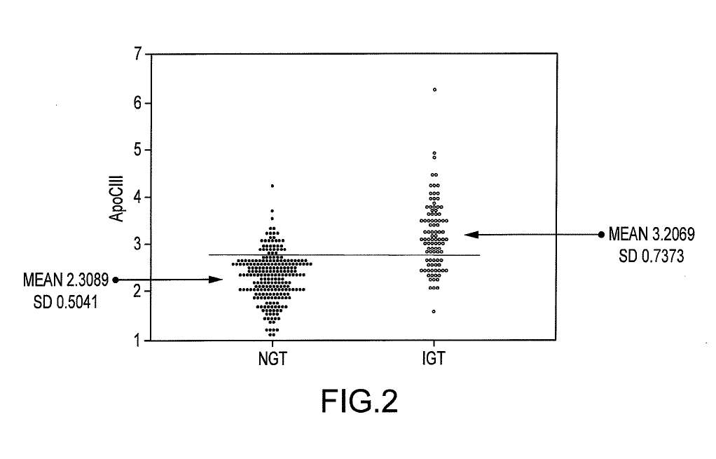

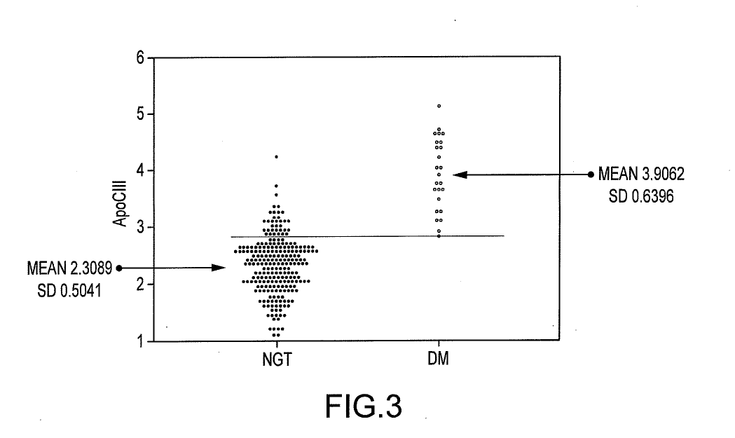

The resulting mass spectrometry data showed the ability to discriminate between samples that originate from NGT patients and IGT/DM patients. Using total ApoCIII as a biomarker for IGT, a cut-off value of 2.6090 was established for IGT determination as shown in FIG. 2. The mean value and one standard deviation of each population are also denoted in FIG. 2. Using total ApoCIII as a biomarker for DM, a cut-off value of 2.7890 was established for DM determination as shown in FIG. 3. The mean value and one standard deviation of each population are also denoted in FIG. 3.

The clinical sensitivity and specificity for ApoCIII were also determined. These values are presented below in Table 1.

| | | | |
|---|---|---|---|
| Biomarker | Sensitivity | Specificity | Cut-off Value |

IGT

| | | | |
|---|---|---|---|
| ApoCIII | 76.0% | 75.7% | 2.6090 |
| ApoCIII(1) | 82.3% | 83.4% | 1.6600 |

DM

| | | | |
|---|---|---|---|
| ApoCIII | 100% | 85.5% | 2.7890 |
| ApoCIII(1) | 100% | 94.5% | 1.9140 |

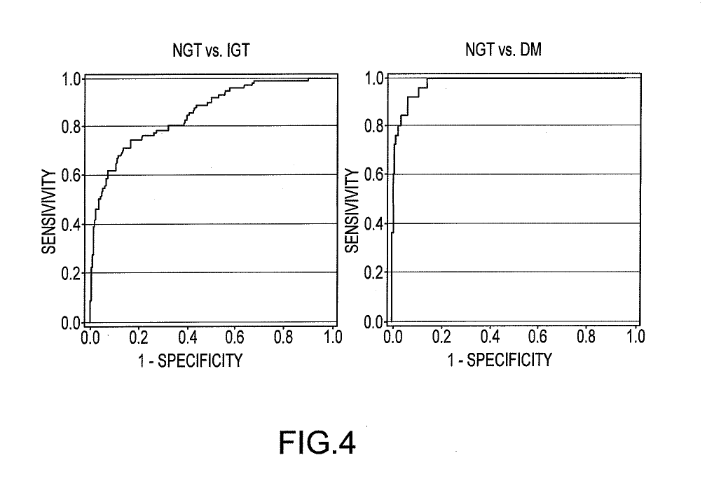

The accuracy of a test to discriminate diseased cases from normal cases is elevated using Receiver Operating Characteristic (ROC) curve analysis. For comparative purposes, the receiver operator characteristic (ROC) curves of the total ApoCIII for IGT and DM were also plotted. These plots are shown in FIG. 4. The displayed plots illustrate the clinical utility of the ApoCIII biomarker in determining/diagnosing both IGT and DM. The calculated areas under the curve for each plot are 0.8492 and 0.9796, respectively.

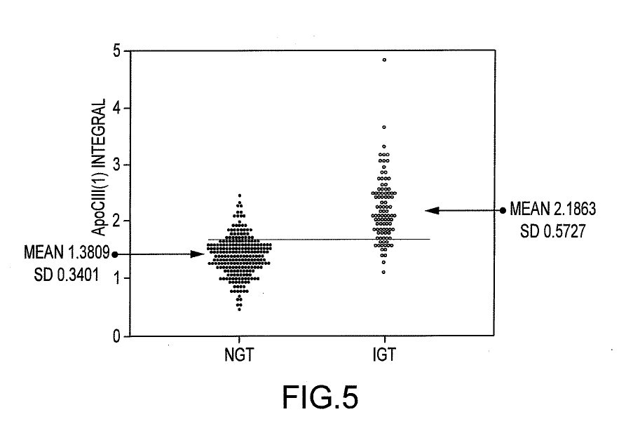

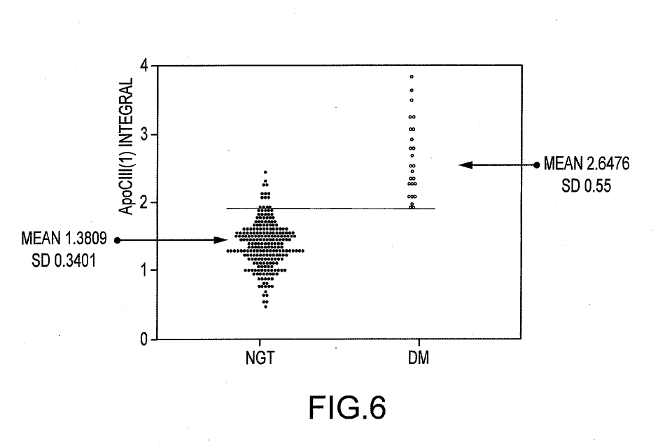

Mass spectrometric immunoassay measurement data of the ApoCIII(1) variant in the samples was then used to perform the same data evaluation. Using ApoCIII(1) as a biomarker for IGT, a cut-off value of 1.6600 was established between the two populations for IGT determination as shown in FIG. 5. The mean value and one standard deviation of each population are also given in FIG. 5. FIG. 6 shows a similar dot histogram, but instead displays ApoCIII(1) abundance within the NGT and DM samples. Using ApoCIII(1) as a biomarker for DM, a cut-off value of 1.9141 was established to differentiate the two groups for DM determination. The mean value and one standard deviation are also denoted in FIG. 6.

The analysis of the ApoCIII(1) variant showed improved clinical utility over the application of the total ApoCIII values. The clinical sensitivity and specificity of the ApoCIII(1) variant as compared to total ApoCIII values is shown in Table 1 above.

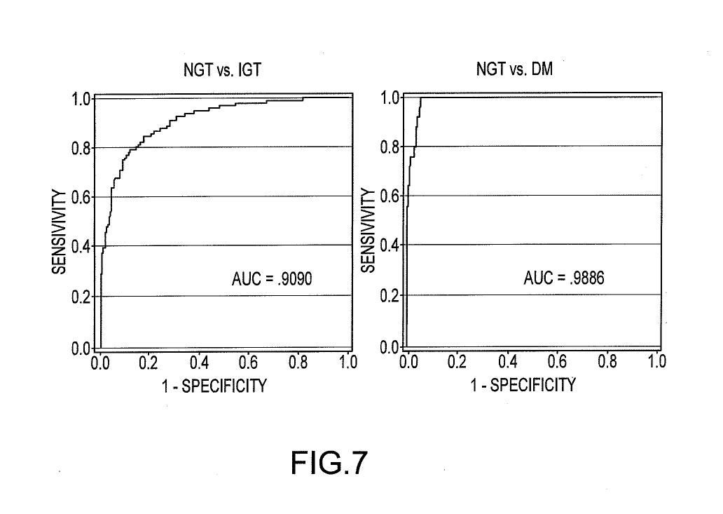

ROC curves for ApoCIII(1) for IGT and DM were also plotted and are shown in FIG. 7. The curves display the clinical utility of the ApoCIII(1) biomarker in determining/diagnosing both IGT and DM. The calculated areas under the curve for each plot are 0.9090 and 0.9886, respectively.

Finally, an algorithm-assisted bio-statistical evaluation of the ApoCIII(1) variant dataset was performed, resulting in improved clinical sensitivity and specificity for determining/diagnosing both IGT and DM. This data is presented below in Table 2.

| | | |
|---|---|---|
| Biomarker | Sensitivity | Specificity |

IGT

| | | |
|---|---|---|
| ApoCIII(1) | 93.6% | 84.3% |

DM

| | | |
|---|---|---|
| ApoCIII(1) | 98.7% | 95.8% |

All of the analyses set forth and/or described above can also be performed on other ApoCIII variants and/or applied to combinations of various observed ApoCIII variants to determine additional biomarkers or groupings of biomarkers for determining IGT, DM and their related conditions.

Exemplary embodiments of the invention are described above in the Drawings and Description of Exemplary Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration, and description. It is not intended to be exhaustive or to limit the invention to the precise form disclosed, and many modifications and variations are possible in the light of the above teachings. Exemplary embodiments were chosen and described in order to best explain the principles of the invention and its practical application and to enable others skilled in the art to best utilize the invention in various embodiments and with various modifications as are suited to the particular use contemplated.

1. A method for at least one of determining, diagnosing, and monitoring at least one of type 2 diabetes, pre-diabetes, insulin resistance, and their related conditions in one or more subjects which includes the step of analyzing at least one of ApoCIII and its variants.

2. The method of claim 1 wherein the step of analyzing ApoCIII and its variants includes the step of analyzing at least one of ApoCIII(0), ApoCIII(1), and ApoCIII(2).

3. The method of claim 1 wherein the step of analyzing at least one of ApoCIII and its variants comprises the step of performing at least one of mass spectroscopy, chromatography, an affinity reaction, gel electrophoresis, centrifugation, chemical methodology, and staining methodology.

4. The method of claim 1 wherein the step of analyzing ApoCIII comprises the step of performing a single assay.

5. The method of claim 4 further comprising the step of performing single mass spectrometry.

6. A method for diagnosing pre-diabetes in a subject comprising the steps of detecting a level of at least one of ApoCIII and its variants in a biological sample of the subject and diagnosing said subject as having pre-diabetes when the level of said at least one of ApoCIII and its variants is modulated by a statistically significant amount from a level present in one or more subjects with normal glucose tolerance.

7. The method of claim 6 wherein the step of diagnosing said subject as having pre-diabetes comprises the step of diagnosing said subject as having pre-diabetes when the level of said at least one of ApoCIII and its variants is increased by a statistically significant amount over a level present in one or more subjects with normal glucose tolerance

8. The method of claim 6 wherein the step of detecting a level of at least one of ApoCIII and its variants comprise the step of detecting a level of ApoCIII(1) and the step of diagnosing said subject as having pre-diabetes comprises the step of diagnosing said subject as having pre-diabetes when the level of ApoCIII(1) is modulated by a statistically significant amount from a level of ApoCIII(1) present in one or more subjects with normal glucose tolerance.

9. The method of claim 8 wherein the step of diagnosing said subject as having pre-diabetes comprises the step of diagnosing said subject as having pre-diabetes when the level of ApoCIII(1) is increased by a statistically significant amount over a level of ApoCIII(1) present in one or more subjects with normal glucose tolerance.

10. A method for diagnosing type 2 diabetes in a subject comprising the steps of detecting a level of at least one of ApoCIII and its variants in a biological sample of the subject and diagnosing said subject as having type 2 diabetes when the level of said at least one of ApoCIII and its variants is modulated by a statistically significant amount from a level present in one or more subjects with normal glucose tolerance.

11. The method of claim 10 wherein the step of diagnosing said subject as having type 2 diabetes comprises the step of diagnosing said subject as having type 2 diabetes when the level of said at least one of ApoCIII and its variants is increased by a statistically significant amount over a level present in one or more subjects with normal glucose tolerance.

12. The method of claim 10 wherein the step of detecting a level of at least one of ApoCIII and its variants comprise the step of detecting a level of ApoCIII(1) and the step of diagnosing said subject as having type 2 diabetes comprises the step of diagnosing said subject as having type 2 diabetes when the level of ApoCIII(1) is modulated by a statistically significant amount from a level of ApoCIII(1) present in one or more subjects with normal glucose tolerance.

13. The method of claim 12 wherein the step of diagnosing said subject as having type 2 diabetes comprises the step of diagnosing said subject as having type 2 diabetes when the level of ApoCIII(1) is increased by a statistically significant amount over a level of ApoCIII(1) present in one or more subjects with normal glucose tolerance.

14. A method for evaluating a therapeutic treatment for at least one of type 2 diabetes, pre-diabetes, insulin resistance and their related conditions in one or more subjects which includes the step of monitoring at least one of ApoCIII and its variants in a biological sample from said one or more subjects.

15. The method of claim 14 wherein the step of monitoring at least one of ApoCIII and its variants includes the step of monitoring at least one of ApoCIII(0), ApoCIII(1), and ApoCIII(2).

16. A method for identifying a therapeutic treatment for at least one of type 2 diabetes, pre-diabetes, insulin resistance and their related conditions which includes the step of determining whether the therapeutic treatment can modulate activity or concentration of at least one of ApoCIII and its variants within a subject.

17. The method of claim 16 wherein the step of determining whether the therapeutic treatment can modulate activity or concentration of at least one of ApoCIII and its variants includes the step of determining whether the therapeutic treatment can modulate activity or concentration of at least one of ApoCIII(0), ApoCIII(1), and ApoCIII(2).
